(19) United States
(12) Patent Application Publication
(10) Pub. No.: US 2022/0195359 A1
(43) Pub. Date: Jun. 23, 2022

(54) CULTIVATION SYSTEMS AND METHODS FOR LARGE-SCALE PRODUCTION OF CULTURED FOOD

(71) Applicant: ALEPH FARMS LTD., Rehovot (IL)

(72) Inventors: Neta LAVON, Ness Ziona (IL); Moshe AVIV, Ness Ziona (IL); Yoav ROTH, Ashdod (IL); Didier TOUBIA, Raanana (IL)

(21) Appl. No.: 17/607,483

(22) PCT Filed: Apr. 30, 2020
(86) PCT No.: PCT/IL2020/050484

§ 371 Date: Oct. 29, 2021

Related U.S. Application Data
(60) Provisional application No. 62/841,864, filed on May 2, 2019.

(51) Int. Cl. C12M 3/00; A23L 13/00; C12N 5/077; C12N 5/071; C12M 1/12; C12M 1/34; C12M 1/36; C12M 3/06; C12M 1/00; C12M 1/26; C12N 5/00

(52) U.S. Cl. CPC C12M 21/08; C12N 2513/00; C12N 5/0653; C12N 5/0658; C12N 5/069; C12M 25/14; C12M 41/44; C12M 41/48; C12M 27/16; C12M 41/12; C12M 41/26; C12M 41/34; C12M 41/32; C12M 23/58; C12M 33/14; C12M 23/26; C12M 23/14; C12N 5/0062; C12N 5/0697; A23V 2002/00; A23L 13/00

(57) ABSTRACT

Systems and methods for producing cultured food products such as cultured meat in a form of meat cut or offal are provided, comprising growing non-human-animal adherent cells on edible scaffold(s) in a cultivation system. The cultivation system typically comprises a plurality of cell culture bioreactors receiving medium at a controlled flow rate adjusted to nourish the non-human-animal adherent cells.

### FIELD OF THE INVENTION

The present invention relates to the production of cultured food products, particularly cultured meat, on a commercial scale, particularly to the manufacturing of cultured meat in the form of meat cuts or offal.

### BACKGROUND OF THE INVENTION

In the past few decades there is an increased interest in food products for human consumption which provide eating experience and nutritional value at least comparable to that of meat, without the environmental hazards and moral issues associated with animal-based meat. This interest promotes the search for systems, methods and compositions for producing cultured meat (also referred to as cell-based meat, clean meat, cultivated meat and slaughter free meat) by both the food industry and the scientific community.

The challenges in manufacturing cultured meat products include production of large quantities of cells and, once obtained, producing products having sensory qualities appealing to the consumers, including visible appearance, texture, flavor and aroma. A further challenge is to scale-up the production processes in order to manufacture the meat products in large quantities suitable for human consumption. A particularly challenging mission is the direct production of a meat portion suitable for serving, rather than separate cultured meat aggregates or layers that need to be fused or connected in order to obtain a meat portion suitable for serving.

Attempts for producing large quantities of mammalian cells, and to adhering the cells to a substance have been taken in the pharmaceutical research and industry, for example for producing stem cells for therapeutic use and for tissue and organ grafting. For examples U.S. Pat. No. 6,911,201 and US Application Publication No. 2010/0233130 disclose methods of producing undifferentiated hemopoietic stem cells using a stationary phase plug-flow bioreactor. In some embodiments, the methods comprise seeding undifferentiated hemopoietic stem cells or progenitor cells into a stationary phase plug-flow bioreactor in which a three-dimensional stromal cell culture has been pre-established on a substrate in the form of a sheet, the substrate including a non-woven fibrous matrix forming a physiologically acceptable three-dimensional network of fibers. International (PCT) Application Publication No. WO 2008/152640 discloses methods of transplanting the three-dimensional stromal cell culture comprising hematopoietic stem cells into a recipient.

U.S. Pat. No. 9,127,242 discloses a single-use, single or multiple tissue, organ, and graft bioreactor and environmental control system. In some embodiments, growing vascular grafts on scaffold tubes is disclosed.

U.S. Pat. No. 9,987,394 discloses a prosthetic implant and methods for producing the same in a bioreactor. The prosthetic implant comprises a biocompatible three-dimensional scaffold and at least two cell types selected from the group consisting of osteoblasts, osteoclasts, and endothelial cells or progenitors thereof.

US Patent Application Publication No. 2011/0287508 discloses bioreactors and methods of using them to produce tissue engineered products or culture cells. More particularly a tissue and cell culture method is disclosed, based upon an expanded bed bioreactor in which an initial resting bed of particles on which or in which cells are attached, encapsulated or immobilized have a fluid passed upwards through the bed to form an expanded bed in which the fluid acts to separate the particles, i.e. under plug flow conditions to enable the relative positions of the particles to be maintained during the step of culturing the cells to form tissue and helps to reduce collisions between particles and turbulent flow or convective mixing.

International (PCT) Application Publication No. WO 2013/016547 discloses engineered meat products formed as a plurality of at least partially fused layers, wherein each layer comprises at least partially fused multicellular bodies comprising non-human myocytes and wherein the engineered meat is comestible.

International (PCT) Application Publication No. WO 2013/116446 discloses methods of generating tubular, bioengineered, smooth muscle structures as well as bioengineered tissue for tubular organ repair or replacement. The methods can include the steps of obtaining smooth muscle cells; culturing the muscle cells to form a smooth muscle cell construct of directionally oriented smooth muscle cells; disposing the smooth muscle cell construct around a tubular scaffold; and culturing construct and scaffold in a growth media until a smooth muscle cell structure is achieved.

International (PCT) Application Publication No. WO 2015/038988 discloses edible microcarriers, including microcarrier beads, microspheres and microsponges, appropriate for use in a bioreactor to culture cells that may be used to form a comestible engineered meat product.

International (PCT) Application Publication No. WO 2018/011805 discloses a system for growing cells comprising a bioreactor chamber for growing the cells, a delivery system delivering a perfusion solution to the bioreactor chamber for perfusion of the perfusion solution through the cells, a dialysis system having a dialyzer, a dialysate for performing a dialysis and a filter for reducing ammonia content in said dialysate, and a controller that circulates the perfusion solution through the dialyzer and the dialysate through the filter.

International (PCT) Application Publication No. WO 2018/189738 discloses a method of producing a hybrid foodstuff. The method comprises combining a plant-originated substance with an amount of cultured animal cells so as to enhance a meat organoleptic and/or meat nutritional property in the hybrid foodstuff, wherein the animal cells do not form a tissue, and wherein the amount of the animal cells and substances thereof is below 30% (w/w) of the hybrid foodstuff.

International (PCT) Application Publication No. WO 2018/227016 discloses systems and methods for producing cell cultured food products. The cultured food products include sushi-grade fish meat, fish surimi, foie gras, and other food types. Various cell types are utilized to produce the food products and can include muscle, fat, and/or liver cells. The cultured food products are grown in pathogen-free culture conditions without exposure to toxins and other undesirable chemicals.

International (PCT) Application Publication No. WO 2019/016795 discloses a method for producing an edible composition, comprising incubating a three-dimensional porous scaffold and a plurality of cell types comprising: myoblasts or progenitor cells thereof, at least one type of extracellular matrix (ECM)-secreting cells and endothelial cells or progenitor cells thereof, and inducing myoblasts differentiation into myotubes.

Nowhere is it disclosed or suggested to produce a cultured food product such as cultured meat by growing animal cells on an edible scaffold within a bioreactor such that a food product comprising the scaffold and a tissue formed from the cells is produced.

There is a need for systems and methods for producing cultured food products such as cultured meat on a commercial scale, providing a cost-effective, fast and simple manufacture of cultured meat products.

### SUMMARY OF THE INVENTION

The present invention provides systems and methods for producing cultured food products, particularly cultured meat, on a commercial scale.

The systems and methods of the present invention comprise growing non-human-animal-derived adherent cells on at least one scaffold in a cultivation system comprising at least one cell culture bioreactor, typically a plurality of cell culture bioreactors. The system of the invention advantageously can provide a controlled flow of a well-defined medium to a plurality of cell culture bioreactors, wherein the medium feed the cells that grow on the scaffold, while not disturbing the cell adherence to the scaffold. According to the present invention, the flow rate and medium composition are preferably adapted to the growth rate and/or growth phase of the cells. Typically, the cultivation system delivers the medium to the plurality of cell culture bioreactors at a uniform flow rate, but adjustment of the flow rate for separate cell culture bioreactors is also enabled by the system of the invention.

The cultured food product according to the present invention comprises at least one three-dimensional, porous, edible scaffold and a tissue formed on and within the scaffold from animal cells seeded of the scaffold. Advantageously and in contrast to hitherto described methods for producing cultured meat, the systems and methods of the present invention produce a meat portion, rather than separate aggregates or small pieces that need to be connected in order to produce a meat portion suitable for serving. In addition, the system of the present invention enables the production of readily packed, sterile cultured food product.

According to one aspect, the present invention provides a cultivation system for producing a cultured food product, comprising:

(a) one or more cell culture bioreactors containing therein two or more types of non-human-animal adherent cells seeded on at least one three-dimensional porous edible scaffold; and

(b) a delivery system configured to deliver medium into the one or more cell culture bioreactors in a controlled flow rate, wherein the flow rate is adjusted to nourish the cells seeded on the at least one three-dimensional porous edible scaffold.

According to certain embodiments, the controlled flow rate is adjusted to prevent bubble formation in the one or more cell culture bioreactor.

According to some embodiments, the controlled flow rate enables circulation of the medium within the bioreactor in a plug-flow manner.

In some embodiments, the system further comprises one or more rockers for radially mixing medium in the one or more cell culture bioreactors. Without wishing to be bound by any specific theory or mechanism of action, the radial mixing contributes to the effective distribution of the cells over the scaffold at seeding and adherence of the cells to the scaffold surface, promoting the cell growth, and to the production of the cultured food product.

In some embodiments, the system further comprises one or more temperature-control elements for controlling the temperature within said one or more cell culture bioreactors. In some embodiments, the system comprises one or more heating elements around the one or more cell culture bioreactors for controlling the temperature within said one or more cell culture bioreactors.

In some embodiments, the system comprises a plurality of cell culture bioreactors. According to these embodiments, the delivery system is configured to deliver the medium to each of the plurality of cell culture bioreactors separately at a controlled flow rate. The delivery of a uniform medium, at a controlled flow rate to the plurality of the cell culture bioreactors is a significant advantage of the cultivation system of the present invention, providing for cost-effective, reproducible large scale production of cultured food products. The medium composition can advantageously be adapted to the cell growth phase. According to certain embodiments, the medium composition is adapted to support the cell proliferation phase. According to some embodiments the medium composition is adapted to support the cell differentiation phase. According to yet further embodiments, the medium composition is adapted to support the cell stationary growth phase.

According to certain embodiments, the controlled flow rate enables circulation of the medium within each of the plurality of cell culture bioreactors in a plug-flow manner.

In some embodiments, each of the plurality of cell culture bioreactors is separately mounted on a rocker. According to some embodiments, each of the plurality of cell culture bioreactors is separately controlled by a temperature-controlling element. In some particular embodiments, each of the plurality of cell culture bioreactors is separately mounted on a rocker and wrapped by a heating element.

In some embodiments, the system further comprises one or more sensors for measuring in the medium at least one parameter selected from the group consisting of liquid level, temperature, pH, dissolved oxygen, concentration of one or more nutrients, and concentration of one or more undesired compounds. Each possibility represents a separate embodiment of the present invention. According to certain embodiments the undesired compound is selected from the group consisting of ammonia, lactate, acetic acid and the like. Each possibility represents a separate embodiment of the present invention.

In some embodiments, the system further comprises a control unit in operative communication with the one or more sensors, configured to receive measurements of the at least one parameter and adjust said at least one parameter based on the measurement.

In some embodiments, the system further comprises:
(i) a medium reservoir for supplying cell growth medium into the cultivation system;
(ii) a treatment vessel configured to: receive medium; measure in the medium at least one parameter selected from the group consisting of liquid level, temperature, pH, dissolved oxygen, concentration of one or more nutrients, and concentration of one or more undesired compounds; and adjust the at least one parameter based on the measurement, wherein the delivery system is further configured to circulate medium from the one or more cell culture bioreactor into the treatment vessel.

According to certain embodiments, the delivery system is further configured to circulate medium from the treatment vessel into the one or more cell culture bioreactors.

According to certain embodiments, the system further comprises a dialysis system having a dialyzer and a dialysate, configured to remove undesired compounds from the medium, wherein the delivery system is further configured to circulate medium from the one or more cell culture bioreactor or the treatment vessel into the dialysis system and subsequently into said treatment vessel.

In some embodiments, the system comprises a dialysis system in which the dialysate flows out of the dialyzer following dialysis as waste.

In some embodiments, the treatment vessel comprises: an impeller; one or more sensors for measuring the at least one parameter; one or more ports configured for addition of at least one of nutrients, neutralizing agent for neutralizing undesired compounds, and two or more types of non-human-animal adherent cells; a heat exchanger; an oxygenator; and a pH control unit.

In some embodiments, the system further comprises a sensing unit configured to measure in the medium at least one parameter selected from the group consisting of temperature, pH, dissolved oxygen, concentration of one or more nutrients and concentration of one or more undesired compounds. Each possibility represents a separate embodiment of the present invention.

According to certain exemplary embodiments, the sensing unit is configured to measure in the medium a combination of parameters comprising temperature, pH, dissolved oxygen, one or more nutrients, and one or more undesired compounds.

In some embodiments, the system further comprises a control unit in operative communication with the treatment vessel and optionally with the sensing unit, for controlling the adjustment of the at least one parameter. According to some embodiments, the control unit is in operative communication with the treatment vessel for controlling the medium temperature. In some embodiments, the medium temperature in the treatment vessel is adjusted to be about the temperature of the cell culture bioreactors.

In some embodiments, the control unit is further in operative communication with the delivery system, for controlling the flow rate and the composition of the medium in the cultivation system. According to certain embodiments, the flow rate is controlled according to the growth rate and/or the growth phase of the cells. According to certain embodiments, the control unit is further in operative communication with the treatment vessel, for controlling the medium composition according to the growth phase of the cells.

In some embodiments, the system is operated to deliver the medium to the one or more cell culture bioreactors in plug-flow manner. According to these embodiments, the plug-flow rate is adapted to the growth rate of the cells.

In some embodiments, the system is operating in a fed-batch mode.

According to certain embodiments, the edible scaffold comprises a protein content of at least 10% by weight based on the dry weight of the scaffold. According to some embodiments, the edible scaffold comprises a protein content of at least 20%, at least 30% or at least 40% by weight based on the dry weight of the scaffold.

In some embodiments, the edible scaffold is of plant, fungal or algal origin.

In some embodiments, the edible scaffold placed within the cell culture bioreactor is sterile.

The two or more types of animal adherent cells are selected as to enable production of a desired meat product. According to certain embodiments, the desired meat product comprises a cell combination mimicking meat cuts, meat portions or offal. According to certain embodiments, the offal is selected from the group consisting of liver, kidney, heart, pancreas, thymus, brain, tongue, and stomach. Each possibility represents a separate embodiment of the present invention.

According to certain embodiments, the two or more types of animal adherent cells are selected from the group consisting of stromal cells, endothelial cells, fat cells, muscle cells, hepatocytes, cardiomyocytes, renal cells, lymphoid cells, epithelial cells, neural cells, ciliated epithelial cells, gut cells, progenitors thereof and combinations thereof. According to certain embodiments the two or more types of animal adherent cells further comprise extracellular matrix (ECM)-secreting cells and progenitors thereof.

In some embodiments, the two or more types of animal adherent cells are selected from the group consisting of muscle cells, extracellular matrix (ECM)-secreting cells, fat cells, endothelial cells, progenitors thereof, and any combination thereof. Each possibility represents a separate embodiment of the present invention.

According to certain embodiments, the system comprises cells from a single animal species origin. In some embodiments, the system comprises cells from a plurality of different species of animal origin. According to certain embodiments, the animal is of a species selected from the group consisting of ungulate, poultry, aquatic animals, invertebrate and reptiles. Each possibility represents a separate embodiment of the present invention.

According to certain embodiments, the ungulate is selected from the group consisting of a bovine, an ovine, an equine, a pig, a giraffe, a camel, a deer, a hippopotamus, or a rhinoceros. According to some embodiments the ungulate is a bovine. According to certain exemplary embodiments, the bovine is a cow.

In some embodiments, the animal adherent cells comprise bovine-derived cells selected from extracellular matrix (ECM)-secreting cells, muscle cells, fat cells, endothelial cells, progenitors thereof and combinations thereof. Each possibility represents a separate embodiment of the present invention.

In some embodiments, the bovine-derived cells are bovine pluripotent stem cells (bPSCs). In some embodiments, the bPSCs are embryonic stem cells. In some embodiments, the bPSCs are bovine induced PSCs (biPSCs).

In some embodiments, the bovine-derived cells are cells differentiated from bovine pluripotent stem cells (bPSC).

In some embodiments, the cultured food product is cultured meat.

In some embodiments, the scaffold is conditioned to have an enhanced capability of adhering non-human-animal cells.

In some embodiments, the cell culture bioreactor has at least an inlet port and an outlet port allowing flow of medium in and out of said cell culture bioreactor.

In some embodiments, the cell culture bioreactor is a flexible bag. According to these embodiments, the bag comprises at least an inlet port and an outlet port allowing flow of medium in and out of said bag.

In some embodiments, the cell culture bioreactor is for a single use.

According to certain embodiments, the cell culture bioreactor in the form of a flexible bag is configured to allow sealing thereof following insertion of the at least one scaffold.

In some embodiments, the inner volume of the cell culture bioreactor is sterile.

According to certain embodiments, the cell culture bioreactor inner face is made of a food-safe material. According to certain embodiments, the bioreactor inner face is made of is made of a material with minimal or none cell adherence capacity. According to some embodiments, the cell culture bioreactor is made of a material selected from the group consisting of a material protecting light-sensitive materials from exposure to light, a material essentially impermeable to water vapors and/or oxygen and a combination thereof.

In some embodiments, the cell culture bioreactor in the form of a flexible bag is configured to allow sealing thereof following growth of the cells to form a packaged food product comprising the cultured food product within the flexible bag.

The flexible bag can be made of a single or multiple layers. According to certain embodiments, the flexible bag inner layer is of a food-safe material. According to some embodiments, the inner layer is made of a material with minimal or none cell adherence capacity. According to certain exemplary embodiments, the flexible bag is made from a laminate comprising an inner layer of a food-safe material. According to certain embodiments, the laminate further comprises at least one layer essentially impermeable to water vapor and/or oxygen. According to some embodiments, the laminate comprises at least one layer protecting light-sensitive materials from exposure to light. According to some embodiments, the laminate comprises at least one layer providing support and strength to the flexible bag.

In some embodiments, the flexible bag comprises an inner layer of food-safe polyethylene, a nylon layer and optionally at least one additional polyethylene layer. In some embodiments, the flexible bag further comprises a layer protecting light-sensitive materials from exposure to light.

According to certain embodiments the volume of the cell culture bioreactor is from about 1 liter to about 500 liters. According to certain embodiments, the volume is from about 2 liters to 400 liters, from about 3 liters to 300 liters or from about 3 liters to 200 liters. According to certain exemplary embodiments, the volume of the tissue culture bioreactor is selected from about 3 liters, about 50 liters and about 200 liters. Each possibility represents a separate embodiment of the present invention. According to certain exemplary embodiments, the cell culture bioreactor is a flexible bag having the volumes described herein.

In some embodiments, the delivery system comprises one or more peristaltic pumps.

In some embodiments, the system further comprises one or more bubble traps.

According to another aspect, the present invention provides a cell culture bioreactor for producing a cultured food product, the cell culture bioreactor is in the form of a flexible bag having an inner face of a food-safe material and comprising at least an inlet port and an outlet port allowing flow of medium in and out of the bag, the bag containing therein at least one three-dimensional porous edible scaffold.

According to certain embodiments, the edible scaffold comprises a protein content of at least 10% by weight based on the dry weight of the scaffold.

In some embodiments, the bag is configured to allow seeding cells on the scaffold while the scaffold is within the bag.

According to some embodiments, the total volume of a single bare scaffold or of a plurality of bare scaffolds inserted to the bag is from about 20% to about 95% of the flexible bag volume. It is to be explicitly understood that the bare scaffold volume does not include the volume of the cells/tissue adhered thereon. According to some embodiments, the total volume of a single bare scaffold or of a plurality of bare scaffolds is from about 30% to about 95%, from about 40% to about 95%, or from about 40% to about 80% of the flexible bag volume.

According to certain embodiments, the at least one three-dimensional porous edible scaffold and the inner volume of the flexible bag are sterile.

In some embodiments, the cell culture bioreactor is for single use.

According to certain embodiments, the cell culture bioreactor is configured to allow sealing of the bag following insertion of the at least one scaffold to the cell culture bioreactor.

In some embodiments, the cell culture bioreactor is configured to allow sealing of the bag following growth of cells on the at least one scaffold and production of a cultured food product, to form a packaged food product comprising the cultured food product within the bag.

In some embodiments, the cultured food product is cultured meat.

In some embodiments, the bag comprises multiple layers. According to these embodiments, the inner layer is made of food-safe material.

In some embodiments, the bag has inner layer of food-safe polyethylene.

In some embodiments, the bag has an inner layer of food-safe polyethylene, a nylon layer and optionally an additional polyethylene layer.

In some embodiments, the bag has a layer protecting light-sensitive materials from exposure to light.

In some embodiments, the bag has a layer essentially impermeable to water vapors and/or oxygen.

According to a further aspect, the present invention provides a packaged food product comprising:

a sealed sterile bag comprising an inner face of a food-safe material; and

a cultured meat portion within the bag, substantially filling the entire inner volume of said bag, the cultured meat portion comprising a cellular tissue comprising a plurality of animal adherent cell types attached to at least one edible three-dimensional porous scaffold.

According to certain embodiments, the three-dimensional porous edible scaffold comprises a protein content of at least 10% by weight based on the dry weight of the scaffold. According to some embodiments, the three-dimensional porous edible scaffold comprises a protein content of at least 20%, at least 30% or at least 40% by weight based on the dry weight of the scaffold.

In some embodiments, the bag comprises multiple layers. According to these embodiments, the inner layer is of food-safe material, wherein the food-safe material is not cell adherent.

According to certain embodiments, the bag comprises an inner layer of food safe polyethylene.

In some embodiments, the bag comprises an inner layer of food-safe polyethylene, a nylon layer and optionally an additional polyethylene layer.

In some embodiments, the bag further comprises a layer protecting light-sensitive materials from exposure to light.

In some embodiments, the bag comprises a layer essentially impermeable to water vapors and/or oxygen. In some embodiments, the plurality of animal adherent cell types is selected from the group consisting of stromal cells, endothelial cells, fat cells, muscle cells, hepatocytes, cardiomyocytes, renal cells, lymphoid cells, epithelial cells, neural cells, ciliated epithelial cells, gut cells, extracellular matrix (ECM)-secreting cells progenitors thereof and any combination thereof. Each possibility represents a separate embodiment of the present invention.

In some embodiments, the plurality of animal adherent cell types is selected from connective tissue cells, muscle cells, fat cells and endothelial cells. Each possibility represents a separate embodiment of the present invention.

According to certain embodiments, the cells are of a non-human-animal selected from the group consisting of bovine, poultry, aquatic animals, invertebrate and reptiles. Each possibility represents a separate embodiment of the present invention. According to certain exemplary embodiments, the non-human-animal cells are bovine cells.

According to certain embodiments, the cultured meat portion is sterile.

According to a further aspect, the present invention provides a method for producing a cultured food product on a commercial scale comprising:

(i) seeding two or more types of non-human animal adherent cells on at least one scaffold disposed within a cell culture bioreactor comprising a cell growth medium, wherein the at least one scaffold is a three-dimensional porous edible scaffold;

(ii) delivering cell growth medium into the cell culture bioreactor in a controlled flow rate and adjusting the flow rate to nourish the cells seeded on the at least one scaffold; and

(iii) growing the cells until a desired tissue mass is obtained, thereby obtaining a cultured food product.

According to certain embodiments, the method further comprises circulating cell growth medium from the cell culture bioreactor to a treatment vessel and/or a dialysis system and subsequently back into the cell culture bioreactor.

According to certain embodiments, the method further comprises adding nutrients to the medium if the concentration of one or more nutrients becomes insufficient, and optionally adding one or more neutralizing agents for neutralizing undesired compounds created along the process. According to certain embodiments, the one or more nutrients and/or neutralizing is added to the medium wherein said medium is within the treatment vessel.

According to certain embodiments, seeding is of single cells. According to other embodiments, seeding is of cell aggregates.

According to certain embodiments, the method further comprises radially rotating the cell culture bioreactor after seeding of the cells to allow adhesion of the cells onto the scaffold.

According to certain embodiments, the controlled flow rate is adjusted to prevent cell detachment from said scaffold and/or bubble formation in the cell culture bioreactor.

According to certain embodiments, the controlled flow rate is adjusted to maintain the cells at a required cell phase. According to certain embodiments, the cell phase is selected from the group consisting of cell proliferation, cell differentiation and cell stationary growth phase. Each possibility represents a separate embodiment of the present invention.

According to certain embodiments, seeding step (i) is repeated at least once throughout the producing the cultured food product.

According to certain embodiments, the three-dimensional porous edible scaffold comprises a protein content of at least 10% by weight based on the dry weight of the scaffold. According to some embodiments, the three-dimensional porous edible scaffold comprises a protein content of at least 20%, at least 30% or at least 40% by weight based on the dry weight of the scaffold. Each possibility represents a separate embodiment of the present invention.

According to certain embodiments, the flow rate is adjusted according to the cell growth rate and/or the cell growth phase. According to some embodiments, the medium composition is adjusted to the cell growth phase. According to certain embodiments, the growth phase is selected from the group consisting of cell proliferation, cell differentiation, and cell stationary growth. Each possibility represents a separate embodiment of the present invention.

The system of the present invention advantageously enables monitoring the medium composition throughout the cell growth and formation of cultured food product. The present invention shows that decrease in glucose concentration and/or increase in lactate concentration in the cell culture medium are reliable parameters reflecting the cell growth rate. According to certain embodiments, the glucose and/or lactate concentration are measured by the sensing unit of the system of the invention. According to other embodiments, the glucose and/or lactate concentration are measured in a sample obtained from the cell culture bioreactor via one of its openings.

According to certain embodiments, the method further comprises sampling the cell growth medium and measuring the concentration of glucose and/or lactase in said growth medium. According to certain embodiments, sampling is repeated at least once. According to some currently exemplary embodiments, sampling is performed every day.

According to certain embodiments, the cells are grown to reach a mass for forming the desired cultured food product.

In some embodiments, the cells are grown until glucose uptake rate (GUR) becomes substantially constant.

In some embodiments, the cells are grown for 5-14 days.

In some embodiments, the method further comprises washing the food product in a water-based solution to remove the growth medium.

According to certain embodiments, the inner volume of the cell culture bioreactor comprising the at least one scaffold, cells and growth medium is kept sterile throughout the process.

In some embodiments, the cell culture bioreactor is in the form of a flexible bag for single use.

In some embodiments, the method further comprises sealing the bag after the cells reach stationary phase to obtain a packaged food product, the packaged food product comprising the sealed bag and the cultured food product within said bag.

According to certain exemplary embodiments, the sealing comprises removing residual medium or washing solution and all gases in the bags using vacuum.

According to another aspect, the present invention provides a cultured food comprising a cellular tissue comprising a plurality of non-human animal adherent cell types attached to at least one three-dimensional porous scaffold, produced by the method of the present invention.

According to certain embodiments, the three-dimensional porous edible scaffold comprises a protein content of at least 10% by weight based on the dry weight of the scaffold. According to some embodiments, the three-dimensional porous edible scaffold comprises a protein content of at least 20%, at least 30% or at least 40% by weight based on the dry weight of the scaffold. Each possibility represents a separate embodiment of the present invention.

Other objects, features and advantages of the present invention will become clear from the following description, examples and drawings.

### BRIEF DESCRIPTION OF THE FIGURES

FIG. 1 illustrates a cultivation system for producing a cultured food product according to some embodiments of the present invention.

FIG. 2 illustrates a cultivation system for producing a cultured food product according to additional embodiments of the present invention.

FIG. 3 shows a flow-chart of a production process to produce a portion of cultured meat according to some embodiments of the present invention.

FIG. 4 illustrates a flexible bag bioreactor according to some embodiments of the present invention.

FIG. 5 demonstrates that cells grown on/in a plant-based scaffold within a flexible bag bioreactor are viable for 250 hours after seeding. Arrows indicate glucose additions at time point when glucose concentration in the medium dropped below 4 g/l.

FIG. 6 demonstrates the presence of two cell types—muscle progenitor cells and fibroblasts on a scaffold within a cell bioreactor according to some embodiments of the invention. At the end of the growth process, samples taken from various areas of the scaffold were homogenized and total RNA was extracted and converted into complementary DNA (cDNA). RT-PCR amplification of the genes encoding Pax 7 (marker of muscle progenitor cells) and Collagen type 1 (marker of fibroblasts) was performed. Lane 1 and Lane 4: RT-PCR products of Pax 7 and Collagen type 1 amplification, respectively, in sample obtained from cell-free scaffold. Lane 2 and Lane 5: RT-PCR products of Pax 7 and Collagen type 1 amplification, respectively, in samples obtained from an edge of the scaffold seeded with muscle progenitor cells and fibroblasts. Lane 3 and Lane 6: RT-PCR products of Pax 7 and Collagen type 1 amplification, respectively, in samples obtained from the opposite edge of the scaffold seeded with muscle progenitor cells and fibroblasts.

### DETAILED DESCRIPTION OF THE INVENTION

The present invention provides cultivation systems and methods for large-scale production of cultured food products, particularly cultured meat.

The cultivation systems of the present invention are flow-rate controlled cultivation systems (also termed continuous tubular cultivation systems), in which medium flows through the system in a controlled manner providing one or more cell culture bioreactors with a uniform medium at a rate configured to maintain cell growth within and onto at least one three-dimensional porous edible scaffold placed within the cell culture bioreactor. The controlled flow rate advantageously provides a flow that can feed the cells that grow on/in the scaffold and mimic muscle mass growth in an animal body. According to the present invention, the flow rate is preferably adapted to the growth rate and/or the growth phase of the cells. According to some embodiments, the controlled flow rate is a plug-flow.

The cultured food product according to the present invention comprises the at least one edible scaffold and a tissue formed on the scaffold from non-human-animal cells. Thus, the scaffold is not separated from the cells/tissue but rather forms part of the final food product, which mimics a cut of a slaughtered meat. The present invention discloses for the first time systems and methods for large scale manufacturing of cultured meat products in the form of meat cuts or meat portions. The cultured meat cuts of the present invention comprise meat-like tissues formed from the cultured cells, distinct from cultured meat burger, nugget, sausage or patty.

The terms “comprise”, “comprising”, “includes”, “including”, “having” and their conjugates mean “including but not limited to”.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The term “about” as used herein refers to a variation of a numerical designation of +10% or −10% of the numerical designation.

The terms “animal” and “non-human animal” with reference to cells derived therefrom are used herein interchangeably and refers only to cells of non-human-animals.

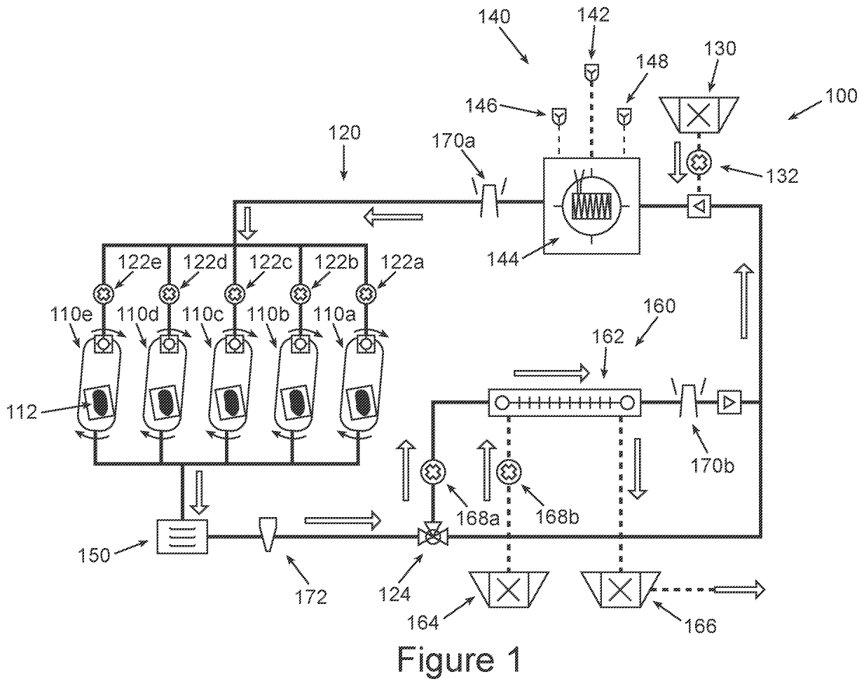

Reference is now made to FIG. 1, which illustrates a cultivation system 100 for producing a cultured food product according to some embodiments of the present invention. Cultivation system 100 comprises a medium reservoir 130, a treatment vessel 140, a plurality of cell culture bioreactors 110a-110e, a sensing unit 150 and a dialysis system 160, all connected via a delivery system 120 that circulates the medium in the cultivation system.

Cultivation system 100 comprises a plurality of cell culture bioreactors 110a-110e arranged in parallel. The cell culture bioreactors in the embodiment illustrated in FIG. 1 are in the form of flexible bags. Each bag is separately mounted on a rocker for radial mixing of the medium in the bag and separately controlled by a temperature-controlling element, for example, wrapped by a heating blanket for controlling the temperature within the bag (not shown). The temperature maintained in the bags is selected according to the types of cells that are seeded on the scaffold. Each bag is further under pressure control. The pressure is typically controlled by at least one exhaust system (not shown). Each bag contains therein at least one edible scaffold 112 capable of adhering non-human-animal cells. In some embodiments, the scaffold is seeded with cells before it is disposed in the cell culture bioreactor. In other embodiments, cells are seeded on the scaffold while the scaffold is within the cell culture bioreactor. In the illustrated embodiment each bag contains therein a single scaffold. In other embodiments, each bag may contain more than one scaffold. In some embodiments, when more than one scaffold is present, each scaffold together with the tissue formed thereon constitutes a separate or one single cultured food product.

Delivery system 120 is configured to deliver uniform medium into each of cell culture bioreactors 110a-110e in a controlled flow rate, for example in a plug-flow manner. Delivery system 120 comprises a plurality of peristaltic pumps 122a-122e, each pump delivers medium into one of cell culture -bioreactors 110a-110e at a rate enabling circulation of the medium within the bioreactor to enable cell growth while preventing cell detachment from the scaffold and/or bubble formation. In certain embodiments, the circulation of the medium within the bioreactor is in a plug-flow manner. Each of cell culture bioreactors 110a-110e separately receives uniform medium, delivered to all cell culture bioreactors 110a-110e by the delivery system 120 at a controlled flow rate enabling cell growth. In certain embodiments, the controlled flow rate enables circulation of the medium within each of the plurality of cell culture bioreactors 110a-110e in a plug-flow manner. Pumps 122a-122e are in operative communication with a control unit (not shown) which controls and adjusts the flow rate to provide the desired medium circulation and prevention of cell detachment from the scaffold and/or bubble formation. In some preferred embodiments, the flow rate is further adjusted according to the growth phase and/or growth rate of the cells on the scaffold. It is to be explicitly understood that each of the pumps 122a-122e can deliver the medium to each of the cell culture bioreactors 110a-110e at the proximal or distal end of said cell culture bioreactors. The change between the cell culture bioreactors 110a-110e proximal and distal ends can be achieved by rotating each of said culture bioreactors, by rotating the connection of each of the pumps 122a-122e (not shown) between the proximal and distal ends of the cell culture bioreactors, or by using two-direction pumps and controlling the flow direction by the control unit (not shown).

The flow rate of the medium is adjusted as to reach each of the cell culture bioreactors and nourish the cells seeded and attached onto the one or more scaffolds within the bioreactor. The flow rate is typically adjusted according to the cells growth rate and particularly according to the cell growth phase, comprising cell proliferation phase, cell differentiation phase and cell stationary phase. According to certain embodiments, the flow rate is adjusted to form a shear force leading to cell differentiation.

A plug-flow within the cell culture bioreactor may be obtained by generating a turbulent flow, rather than laminar flow, when the medium enters the bioreactor. If the flow is sufficiently turbulent then the laminar sublayer caused by the bioreactor's wall is so thin in relation to its diameter such that it is negligible (δ^{s}<<D).

Turbulent flow generation can be predicted using the Reynolds number. For a bioreactor with a round cut, when the Reynold number is less than 2,300 the flow will be laminar, and if it is over 4000 the flow will be turbulent (Reynold numbers between 2,300-4,000 are transit numbers).

For flow in a pipe or a tube (such as a tubular bioreactor according to the present invention), the Reynolds number (“Re”) is generally defined as:

Re=(ρvDH)/μ=(uDH)/v=(QDH)/vA

Q=the volumetric flow rate (m³/s)

D_{H}=the inner diameter of the pipe or tube (m)

v=the kinematic viscosity (v=μ/ρ) (m²/s)

A=the pipe's cross-sectional area (m²)

u=the mean velocity of the fluid (m/s)

μ=the dynamic viscosity of the fluid (Pa·s=N·s/m²=kg/(m·s))

ρ=the density of the fluid (kg/m³)

Alternatively, or additionally, the plug-flow within the cell culture bioreactor may be obtained by adding a suitable mixer (static or dynamic) in the flow line.

Medium reservoir 130 supplies cell growth medium into the cultivation system. Medium reservoir 130 is connected to the cultivation system via a level pump 132, configured to control the flow of medium from the medium reservoir into the cultivation system based on the liquid level inside the cultivation system.

Treatment vessel 140 is configured to receive medium and adjust temperature, pH, dissolved oxygen, concentration of one or more nutrients in the medium, and one or more undesired compounds. Treatment vessel 140 comprises a nutrition addition port 142, a heat exchanger 144, an oxygenator 146 and a pH control unit 148. In some embodiments, the treatment vessel further comprises sensors (not shown) for measuring the aforementioned parameters, namely, temperature, pH, dissolved oxygen, concentration of one or more nutrients, and one or more undesired compounds in the medium. In other embodiments, such as the embodiment illustrated in FIG. 1, the cultivation system comprises a separate sensing unit 150, configured to measure the aforementioned parameters in the medium. In some embodiments, a control unit in operative communication with the sensing unit and the treatment vessel (not shown) controls the adjustment of the parameters as needed based on the measurements. Treatment vessel 140 is further configured to measure liquid level and adjust the liquid level as needed based on the measurement.

Dialysis system 160 is configured to remove undesired compounds such as ammonia and lactic acid from the medium. Dialysis system 160 comprises a dialyzer 162, a fresh dialysate reservoir 164 and a used dialysate reservoir 166.

In the illustrated embodiment, delivery system 120 circulates medium from cell culture bioreactors 110a-110e into the dialysis system and subsequently into the treatment vessel, or, directly into the treatment vessel if no dialysis is needed. The flow of medium into the dialysis system or directly back into the treatment vessel is controlled by a 3-way valve 124. In other embodiments, a dialysis system is not included, and the medium is circulated between the treatment vessel and the cell culture bioreactors.

Dialyzer 162 receives a fresh dialysate from reservoir 164 and spent medium from cell culture bioreactors 110a-110e that needs to be dialyzed to remove undesired compounds that could interfere with the growth of the cells. The flow of the spent medium and fresh dialysate into dialyzer 162 is controlled via pumps 168a and 168b, respectively. In the illustrated embodiment, after dialysis, the dialyzed medium flows to treatment vessel 140 and the used dialysate flows to reservoir 166 and subsequently discarded as waste.

Cultivation system 100 further comprises bubble traps 170a, 170b, to prevent bubble formation within the cell culture bioreactors during operation of the cultivation system. Cultivation system 100 may further comprise sampling point 172.

In some embodiments, production of a cultured food product using cultivation system 100 comprises the following steps:

Seeding of cells: each of cell culture bioreactors 110a-110e, containing therein at least one edible scaffold capable of adhering non-human-animal adherent cells, is mounted on a rocker. Each cell culture bioreactor is filled with a cell growth medium via pumps 122a-122e until the medium fills 5%-80% of the volume of the cell culture bioreactor. Pumps 122a-122e are then shut-off and cells are seeded on the scaffold while it is in the cell culture bioreactor. Alternatively, cells are seeded in treatment vessel 140 and delivered to each of the cell culture bioreactors 110a-110e through delivery system 120. Cells can be seeded as single cells or as cell aggregates. Each cell culture bioreactor is radially rotated by the rocker to maintain the cells in suspension to enhance their distribution and adherence all over the scaffold. Seeding of the cells can be performed once or sequential seeding steps can be performed. According to certain embodiments, the first seeding is performed at the beginning of the cultivation process just after the culture bioreactor is filed with medium and the scaffold is a bare from cells. Sequential seeding steps can be performed any tine thereafter during the cultivation process after cells have been adhered to the scaffold. In certain embodiments, cells are seeded on the at least one scaffold before the scaffold is placed within the cell culture bioreactor. According to these embodiments, each of the cell culture bioreactors comprising the at least one seeded scaffold is filled with a cell growth medium via pumps 122a-122e until the medium fills 5%-80% of the volume of the cell culture bioreactor.

Culturing: following cell adhesion, typically between 6-24 hours after seeding, pumps 122a-122e are activated and circulation of cell growth medium in the cultivation system begins. The medium is delivered into each of cell culture bioreactor 110a-110e in −a controlled flow rate, for example in plug flow. During operation of the cultivation system the flow rate is adjusted to prevent cell detachment from the scaffold and/or bubble formation in each of the cell culture bioreactors. During operation of the cultivation system, the temperature, pH, dissolved oxygen, concentration of one or more nutrients, and optionally concentration of one or more undesired compounds are monitored in the medium. If needed, one or more of these parameters is adjusted in the treatment vessel. For example, if the concentration of a certain nutrient becomes insufficient, the nutrient is supplied into the cultivation system via nutrient addition port 142 in treatment vessel 140. In some embodiments, medium that flows from the cell culture bioreactors is delivered into a dialysis system in order to remove undesired compounds such as ammonia from the medium. The medium is subsequently delivered to the treatment vessel (and again to the cell culture bioreactors). In other embodiments, medium flows directly from the cell culture bioreactors to the treatment vessel (and subsequently to the cell culture bioreactor). The cells are grown in the cell culture bioreactors until the cells reach stationary phase and/or a desired mass and form a cultured food product comprising a tissue formed from the cells and the scaffold(s). In some embodiments, the cells are grown until glucose uptake rate (GUR) becomes substantially constant. In some embodiments, the cells are grown for 5-21 days. When culturing is completed the obtained food product may be washed with a water-based solution, e.g., in saline, to remove the growth medium and optionally add additives to the cultured food product, for example, additives that increase its vitamin content and/or additives that affect its appearance and/or taste.

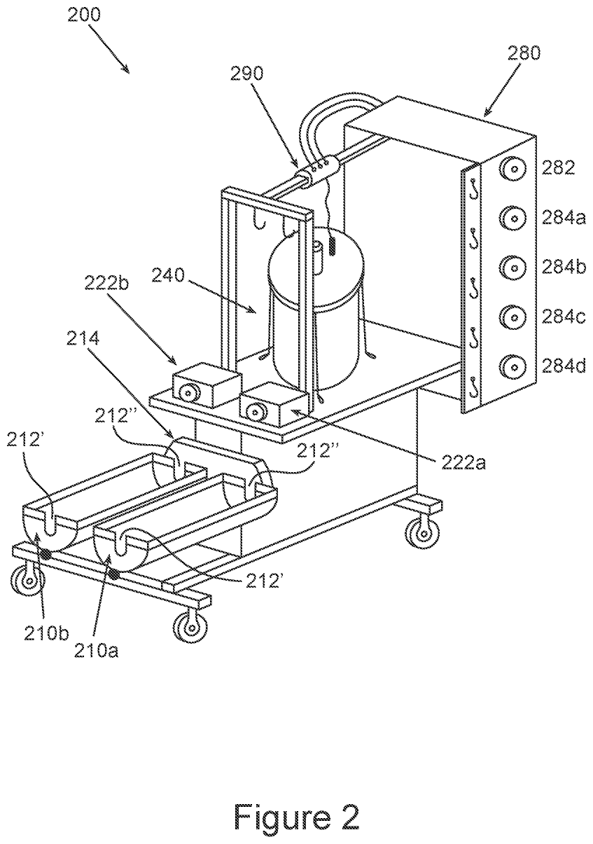

Reference is now made to FIG. 2, which illustrates a cultivation system 200 for producing a cultured food product according to some embodiments of the present invention. Cultivation system 200 comprises a plurality of trays 210a-210b for mounting a plurality of cell culture bioreactors. Each tray has an opening 212 at each end for placing a tubing system of each cell culture bioreactor. Cultivation system 200 further comprises bearing house 214 for force delivery and rocking speed control. Cultivation system 200 further comprises pumps 222a-222b, to be connected to the cell culture bioreactors via a tubing system and deliver medium to the cell culture bioreactors in a controlled flow rate. Cultivation system 200 further comprises a treatment vessel 240 configured to receive medium and to adjust temperature, pH, dissolved oxygen, concentration of one or more nutrients in the medium, and optionally concentration of one or more undesired compounds. Cultivation system 200 further comprises a control unit 280 comprising a base pump 282 for adjusting the pH, a plurality of ingredients, pumps 284a-284c configured to add nutrients and or agents for neutralizing undesired compounds within the medium, and a medium level pump 286. Control unit 280 is connected to treatment vessel 240 via a tubing system (not shown), for controlling pH, supplying nutrients to the medium and maintaining the medium level, as needed during operation of the cultivation system. Control unit 280 and treatment vessel 240 are further connected via a gas manifold 290 of CO₂, O₂ and air, for controlling and supplying CO₂, O₂ and/or air into the medium. In the system illustrated in FIG. 2 the treatment vessel comprises electrodes/sensors for measuring the temperature, pH etc.

Cell Culture Bioreactor

A cell culture bioreactor according to the present invention is a sterile vessel configured to accommodate one or more scaffolds with cells seeded thereon, and allow growing of the cells on and/or within the scaffold to form a cultured food product. In some embodiments, the cell culture vessel is a flexible tubular bag. In some embodiments, the cell culture bioreactor is disposable. The cell culture bioreactor comprises at least an inlet port and an outlet port, to allow flow of medium in and out of the cell culture bioreactor. In some embodiments, the cell culture bioreactor is configured to allow seeding of cells onto the scaffold while the scaffold is inside the cell culture bioreactor.

In some embodiments, a cell culture bioreactor in the form of a flexible bag. Material suitable for forming flexible bag suitable for use as cell culture bioreactor are known in the Art, and can provide the bag with the desired strength, flexibility, and standards of extractables and leachables as required in the food industry.

According to certain embodiments, a cell culture bioreactor in the form of a flexible bag is made from a food-safe material. According to some embodiments, the food safe material is further characterized by minimal or no capacity of cell adherence. In other embodiments, a cell culture bioreactor in the form of a flexible bag is made from a laminate comprising an inner layer of a food-safe material. An example of a food-safe material is a food-safe polyethylene. In some embodiments, a laminate of a cell culture bag according to the present invention further comprises a layer providing support and strength, such as a nylon layer. In some embodiments, the laminate comprises an inner layer of polyethylene and at least one outer nylon layer. In some embodiments, the laminate comprises an inner layer of polyethylene, a nylon layer and at least one additional polyethylene layer. In some embodiments, the laminate comprises at least one layer protecting light-sensitive materials from exposure to light, for example, a layer made from an opaque material, such as an aluminum layer. According to certain embodiments, the laminate comprises at least layer essentially impermeable to water vapor and/or oxygen.

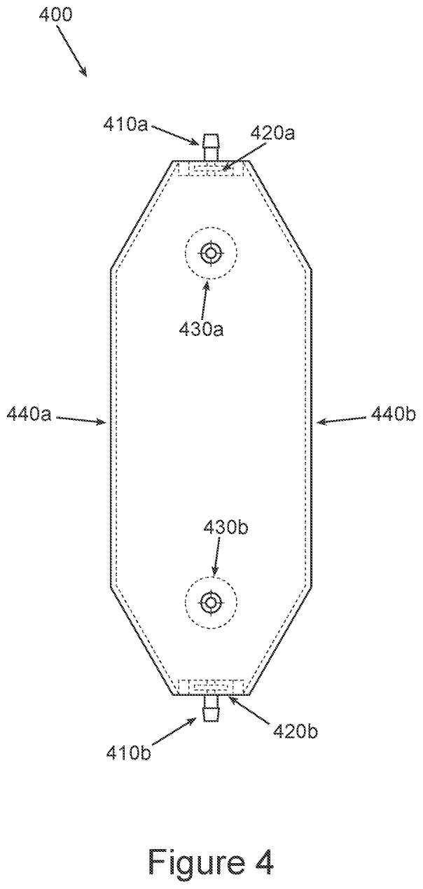

Reference is now made to FIG. 4, which illustrates a culture bioreactor in the form of a flexible bag 400 for seeding two or more types of animal adherent cells on one or more scaffolds disposed within the cell culture bioreactor for obtaining culture food product. The flexible bag 400 is configured as an elongated bag with two end ports 410a-410b located at opposing short faces 420a-420b of the flexible bag 400, with, for example port 410a being an inlet port configured to receive medium from a medium reservoir (for example medium reservoir 130 in FIG. 1) via a delivery system (for example delivery system 120 in FIG. 1) and port 410b configured to deliver medium via, for example the delivery system 120 of FIG. 1 to a treatment vessel (for example treatment vessel 140 of FIG. 1). The flexible bag 400 further comprises one or more openings 430a-430b for seeding the two or more types of animal adherent cells on the one or more scaffolds, and optionally for sampling the culture medium with the flexible bag 400 ay time during the cell growth. An elongated face 440a or 440b may be welded only after the at least one scaffold is inserted to the flexible bag 400 before the bag is mounted on the cultivation system. The volume of the cell culture bioreactor according to the present invention may vary depending on its intended use, for example, whether it is intended to form the package of the cultured food product produced therein, or to be used as a vessel for growing the food product, after which the food product is harvested from the bioreactor. The volume may range, for example, between 1 liter up to 200 liters.

In some embodiments, the cell culture bioreactor in the form of a flexible bag forms the packaging of the cultured food product following its production. According to these embodiments the cells are grown on/in the scaffold in the cell culture bag until desired growth is achieved and a cultured food product comprising the scaffold(s) and cells is formed. The bag is then sealed and disconnected from the cultivation system, to obtain a packaged food product comprising the cultured food product within the bag, substantially filling the entire inner volume of the bag. According to some embodiments, the bag is vacuum-sealed. According to some embodiments, the bag is sealed after the remaining medium is removed and optionally after the food product is washed and the washing solution is removed. According to certain embodiments, the washing solution is water-based solution.

As the growth process is sterile, the resulting packaged food product is sterile and thus have a shelf-life much longer than conventional fresh products such as fresh meat. Furthermore, as the obtained food product is sterile, it may be shipped without the need for cooling, thus significantly reducing the costs involved.

### Scaffold

As used herein, the term “scaffold” refers to a three-dimensional structure comprising a material that provides a surface suitable for adherence/attachment and proliferation of cells. A scaffold may further provide mechanical stability and support. A scaffold may be in a particular shape or form so as to influence or delimit a three-dimensional shape or form assumed by a population of proliferating cells.

The scaffold according to the present invention is a three-dimensional porous substrate made from an edible material suitable for human consumption. In some embodiments, the scaffold material contains at least 10% protein (w/w—dry weight), at least 20%, at least 30%, at least 40%, at least 50%, at least 60%, at least 70%, at least 80%, at least 90% protein (w/w—dry weight). Each possibility represents a separate embodiment of the present invention. In some embodiments, the scaffold is of plant, fugal or algal origin. In some embodiments, the scaffold is of plant or fungal origin.

In some embodiments, the plant/fungi/algae-based three-dimensional porous edible scaffold comprises plant, fungi or algae protein(s), optionally in combination with plant, fungi or algae polysaccharide(s). Each possibility represents a separate embodiment of the present invention.

In some embodiments, the scaffold comprises at least one plant protein optionally with at least one plant polysaccharide, wherein the plant is selected from the group consisting of wheat, soybean, safflower, corn, peanut, peas, sunflower, chickpea, cotton, coconut, rapeseed, potato and sesame. Each possibility represents a separate embodiment of the present invention.

The proteins and optionally polysaccharides can be obtained from any plant part comprising same, including seeds, leaves, roots, steams, tubers, bulbs and the like, and in some embodiments form part of an extract obtained therefrom. In some embodiments, the extract comprises at least 30%, at least 35%, at least 40%, at least 45%, at least 50%, at least 55%, at least 60%, at least 70%, at least 75%, at least 80%, at least 85%, at least 90% or at least 95% protein on a dry weight basis. In some embodiments, the scaffold comprises pure plant protein.

In some embodiments, the scaffold is of fugal origin. In some embodiments, the scaffold material is obtained from edible fungi, typically macro fungi. Any part of the edible fungi can be used, including the mycelia, hyphae and fruit body (sporocarp). In some embodiments, the scaffold material is obtained from an edible mushroom selected from the group consisting of Agaricus bisporus (common mushroom, portobello mushroom), Pleurotus ostreatus (oyster mushroom), Morchella esculenta (morel) and mushrooms of the genera Chanterelle. Each possibility represents a separate embodiment of the present invention.

In some embodiments, the protein or polysaccharide derived from the plant or the fungi or algae comprises a long chain of building blocks. Long chain proteins/polysaccharides provide the scaffold with fibrous texture. The plant or fungi protein can be texturized to a three-dimensional porous scaffold by any method as is known in the art and as described, for example, in WO 2019/016795.

The scaffold according to the present invention supports the growth of cells due to its interconnective pore structure and mechanical properties. The porous structure of the scaffold is essential to allow the cells to penetrate into the depth of the scaffold and allow dispersion to cover it homogenously. Moreover, the interconnected pores allow a liquid flow into the scaffold and promise the nourishment of the cells.

The initial cell seeding density has to be efficient while allowing optimal cell proliferation within the scaffold. The number of the cells to be seeded further depends on the porosity of the scaffold material and its liquid absorption capability. The more the scaffold can absorb, the larger the number of cells that can be seeded. In some embodiments, the number of cells per gr scaffold (dry weight) is in the range of 2×10⁶ to 50×10⁶ cells. In addition, the porosity of the scaffold and the internal organization of scaffold fibers contribute to the retention of the cells within and on the scaffold. The cells can be seeded once or sequential seeding steps throughout the cultivation period can be taken.

Before use, the scaffold is typically sterilized. Sterilization may be performed, for example, by gamma-irradiation, by autoclave, by washing with alcohol or by ethylene oxide (EtO) gas treatment.

In some embodiments, the scaffold is selected from a textured protein and a non-textured protein, optionally further comprising a polysaccharide. In some embodiments, the textured protein is a textured soy protein.

In some embodiments, the scaffold comprises pores with an average diameter ranging from 20 to 1,000 micrometers.

In some embodiments, an average pore diameter of the porous scaffold ranges from 20 micrometers (μm) to 1000 μm, 20 μm to 900 μm, 20 μm to 800 μm, 20 μm to 700 μm, 20 μm to 600 μm, 20 μm to 500 μm, 20 μm to 400 μm, 20 μm to 300 μm, 20 μm to 200 μm, 20 μm to 100 μm, 50 μm to 1000 μm, 50 μm to 900 μm, 50 μm to 800 μm, 50 μm to 700 μm, 50 μm to 600 μm, 50 μm to 500 μm, 50 μm to 400 μm, 50 μm to 300 μm, 50 μm to 200 μm, 50 μm to 100 μm, 100 μm to 1000 μm, 100 μm to 900 μm, 100 μm to 800 μm, 100 μm to 700 μm, 100 μm to 600 μm, 100 μm to 500 μm, 100 μm to 400 μm, 100 μm to 300 μm, 100 μm to 200 μm, 500 μm to 1000 μm, 500 μm to 900 μm, 500 μm to 800 μm, 500 μm to 700 μm, or 500 μm to 600 μm. Each possibility represents a separate embodiment of the present invention.

In some embodiments, coverage % of the plurality of cells is at least 10%, at least 20%, at least 30%, at least 40%, at least 50%, at least 60%, at least 70%, at least 80%, at least 90, or at least 99%. The term “coverage %” refers to the area or volume of the porous scaffold that is in contact with cells throughout the culture process. In some embodiments, coverage % of the plurality of cells is 5-20%, 15-30%, 25-40%, 35-50%, 45-60%, 55-70%, 65-80%, 75-90%, 85-100%, or any range therebetween. Each possibility represents a separate embodiment of the present invention.

The seeding and/or the culturing of cells is performed in the presence of a growth medium. In some embodiment, the growth medium comprises growth factors, small molecules, bioactive agents, nutrients, amino acids, antibiotic compounds, anti-inflammatory compounds, or any combination thereof.

In some embodiments, the scaffold comprises a textured protein. In some embodiments, the textured protein is a textured vegetable protein. In some embodiments, the textured protein is a textured soy protein (e.g., TSP). The term “texture” as used herein regarding to protein refers to a rigid mass, or flexible mass, of individual cells which can be readily formed into various sizes, shapes and configurations and which is non-dispersible in water.

Suitable particulate textured protein materials for use in forming a scaffold of the present invention can consist of from 40% to 100% protein, on a dry weight basis, and from 0% to 60% materials associated with the protein source material or added adjuvant materials. Examples of adjuvant materials are carbohydrates, vitamins, flavors, colorings or others.

Suitable un-textured proteins which can be texturized to form textured particulate protein materials are available from a variety of sources. For example, a source of such proteins is a vegetable protein and certain fungal proteins. Examples of suitable vegetable protein sources are soybeans, safflower seed, corn, peanuts, wheat, wheat gluten, peas, sunflower seed, chickpea, cottonseed, coconut, rapeseed, sesame seed, leaf proteins, gluten, and the like. Proteins of single cell microorganisms such as yeast can also be used.

Generally, if the protein source is a vegetable protein, the protein prior to use is placed in a relatively pure form. Thus, for example if the protein source is soybeans, the soybeans can be solvent extracted, such as with hexane, to remove the oil therefrom. The resulting oil-free soybean material contains about 50% protein.

The soybean material can be processed in a known manner to remove carbohydrates and obtain products with higher levels of protein, for example, soy protein concentrates containing about 70% protein or soy protein isolates containing about 90% or more protein. A variety of processes can be employed to convert the soybean material, concentrate, isolate and other edible protein bearing materials into suitable texturized particulate protein materials, as described in WO 2019/016795.

In some embodiments, the scaffold is conditioned to enhance adherence of the cells.

In some embodiments, a thermo-reversible solidifying agent may be used during the seeding of the cells in order to improve their attachment to the scaffold. In some embodiments, the seeding may include:

(a) incubating a seeding medium comprising a plurality of non-human-animal adherent cell types with the scaffold and a thermo-reversible solidifying agent, wherein the incubation conditions enable solidifying of the medium, thereby forming an essentially semi-solid or solid seeding medium comprising the scaffold and the cells; and

(b) incubating the essentially semi-solid or solid seeding medium under conditions enabling said medium to liquefy.

In some embodiments, when the seeding is carried out in the cell culture bioreactor, the system of the present invention further comprises a cooling system to enable solidification of the medium.

According to certain embodiments, when the cell culture bioreactor in the form of a flexible bag which forms the packaging of the cultured food product following its production, the volume of a single scaffold or the total volume of a plurality of scaffolds inserted to the bag is from 20% to about 99% of the bag inner volume. According to certain embodiments, the volume of the scaffold or plurality of scaffolds is from about 20% to about 95%, 96%, 97% or 98% of the bag inner volume. In some embodiments, the volume of the scaffold or plurality of scaffolds is from about 20% to about 80%, 85%, 86%, 87%, 88%, 89%, 89%, 90%, 91%, 92%, 93%, or 94% of the bag inner volume. In some embodiments, the volume of the scaffold or plurality of scaffolds is from about 25%, 26%, 27%, 28%, 29%, 30%, 40%, 41%, 42%, 43%, 44%, 45%, 46%, 47%, 48%, 49% or 50% to about 95% of the bag inner volume. According to some embodiments, the volume of the scaffold or plurality of scaffolds is from about 40% to about 80% of the bag inner volume.

### Cells

According to the present invention, two or more types of animal adherent cells are seeded on each scaffold. The cells according to the present invention are non-human-animal, non-genetically modified, adherent cells.

To produce cultured meat, the two or more types of non-human-animal adherent cells are selected according to the desired type of meat portion to be produced. The produced meat portion can mimic a cut of slaughtered meat, an offal, or designed for the preparation of a certain dish.

According to certain embodiments, the non-human-animal adherent cells comprise stromal and/or endothelial cells and/or fat cells together with at least one cell type according to the desired final meat product, including muscle cells (meat cuts); hepatocytes (liver); cardiomyocytes (heart); renal cells (kidney); lymphoid and epithelial cells (sweetbread made of thymus and pancreas), neural and neuronal cells (brain); ciliated epithelial (tongue) and stomach cells (tripe).

According to certain embodiments, the non-human-animal adherent cells are selected from the group consisting of muscle cells, extracellular matrix (ECM)-secreting cells, fat cells, endothelial cells, and progenitors thereof. In some embodiments, the two or more types of non-human-animal adherent cells comprise muscle cells or progenitors thereof and at least one additional type selected from the group consisting of ECM-secreting cells, fat cells, endothelial cells, and progenitors thereof. In some embodiments, the non-human-animal adherent cells comprise muscle cells or progenitors thereof, ECM-secreting cells or progenitors thereof, fat cells or progenitors thereof, and endothelial cells or progenitors thereof.

According to certain embodiments, the non-human-animal is selected from the group consisting of ungulate, poultry, aquatic animals, invertebrate and reptiles. Each possibility represents a separate embodiment of the present invention.

According to certain embodiments, the ungulate is selected from the group consisting of a bovine, an ovine, an equine, a pig, a giraffe, a camel, a deer, a hippopotamus, or a rhinoceros. According to some embodiments the ungulate is a bovine. According to certain exemplary embodiments, the bovine is a cow.

In some embodiments, the non-human-animal derived adherent cells to be seeded on the scaffold according to the teachings of the present invention comprise pluripotent stem cells. According to certain embodiments, the non-human-animal derived adherent cells to be seeded comprise bovine-derived pluripotent stem cells (bPSCs). According to certain embodiments, the bPSCs are bovine embryonic stem cells. According to certain embodiments, the bPSCs are bovine induced pluripotent stem cells (biPSCs). The seeded bovine-derived adherent cells are grown under conditions enabling differentiation to the desired cell types. In some particular embodiments, the seeded pluripotent bovine-derived cells are differentiated to muscle cells, ECM-secreting cells, fat cells and/or endothelial cells.

In some embodiments, the non-human-animal derived adherent cells to be seeded on the scaffold according to the teachings of the present invention comprise differentiated cells.

In some embodiments, the non-human-animal cells are obtained by differentiating pluripotent stem cells, for example, bovine-derived pluripotent stem cells (PSCs). According to these embodiments, the process for producing cultured meat comprises expansion and differentiation steps which are carried out before the cells are seeded on the scaffold and incubated in the cultivation system of the present invention. In some embodiments, the process comprises the following steps: (a) seeding the PSCs in a cell culture vessel with expansion medium, the expansion medium is a serum-free, liquid medium comprising a combination of growth factors to form homogenous aggregates that grow with time; (b) splitting the PSCs to four cell culture vessels for differentiation to ECM-secreting cells, myoblast cells, fat cells and endothelial cells; and (c) seeding at least two types of the differentiated cells on at least one scaffold according to the present invention and growing the cells in a cultivation system as described herein.

In some embodiments, the non-human-animal cells are selected from the group consisting of muscle cells, fat cells, stromal cells, fibroblasts, pericytes, endothelial cells and their progenitors. Each possibility represents a separate embodiment of the present invention.

In some embodiments, the plurality of cell types comprises myoblasts and/or progenitor cells thereof and at least one type of extracellular matrix (ECM)-secreting cells.

In some embodiments, the plurality of cell types comprises myoblasts and/or progenitor cells thereof and endothelial cells and/or progenitor cells thereof.

In some embodiments, the plurality of cell types comprises myoblasts and/or progenitor cells thereof, at least one type of extracellular matrix (ECM)-secreting cells and/or progenitor cells thereof and endothelial cells and/or progenitor cells thereof.

In some embodiments, the myoblast progenitor cells are satellite cells.

In some embodiments, the ECM-secreting cells are selected from the group consisting of stromal cells, fibroblasts, pericytes, smooth muscle cells and progenitor cells thereof. Each possibility represents a separate embodiment of the present invention. In some particular embodiments, the ECM-secreting cells are fibroblasts, fibroblast progenitor cells or a combination thereof. Each possibility represents a separate embodiment of the present invention.

In some embodiments, the endothelial cells are selected from the group consisting of skeletal microvascular endothelial cells, aortic smooth muscle cells and a combination thereof. Each possibility represents a separate embodiment of the present invention.

In some embodiments, the plurality of cell types comprises myoblasts, ECM-secreting cells and endothelial cells.

In additional embodiments, the plurality of cell types comprises satellite cells, ECM-secreting cells, and endothelial cells.

In some embodiments, the ratio of the myoblast cells and/or progenitor cells thereof to the ECM-secreting cells is between about 10:1 and about 1:10.

In some embodiments, the ratio of the ECM-secreting cells to the endothelial cells is between about 1:1 and about 1:10.

In some embodiments, the ratio of the satellite cells, the ECM-secreting cells and the endothelial cells is between about 10:1:1 and about 2:1:10.

In some embodiments, the ratio of the satellite cells and the ECM-secreting cells is between about 1:5 to 3:5.

In some embodiments, the ratio between the various cell types in the final product is as follow: 55%-98% of myoblast, 2%-10% of stromal cells, 0%-25% fat cells, 0%-10% endothelial cells.

The seeding medium and the growth medium to be used according to the present invention are those known in the art to be suitable for keeping the viability, proliferation and optionally differentiation of the non-human-animal cells. According to certain exemplary embodiments, an inhibitor of Rho associated protein kinase (Rock) is added at a suitable time to enhance cell survival and overall cell proliferation efficacy.

In some embodiments, the growth medium is a serum-free, animal-derived-component-free, liquid medium for non-human-animal cells enriched with a supplement selected from the group consisting of at least one natural colorant, cyanocobalamin (vitamin B12), iron and any combination thereof, wherein the supplement is in amount sufficient to confer red-brown color to the cells. In some embodiments, the growth medium is characterized by having absorbance at a plurality of wavelengths between about 350 and about 700 nm. In some embodiments, the growth medium further comprises yeast extract, bacterial extract or a combination thereof. Each possibility represents a separate embodiment of the present invention.

In some embodiments, the natural colorant is selected from the group consisting of an extract obtained from at least one non-mammal organism, at least one carotenoid, at least one betalain and any combination thereof. Each possibility represents a separate embodiment of the present invention.

In some embodiments, the growth medium further comprising at least one supplement selected from the group consisting of folate, zinc, selenium, vitamin D, vitamin E, Coenzyme Q10, at least one unsaturated fatty acid, and any combination thereof. Each possibility represents a separate embodiment of the present invention.

In some embodiments, the vitamin D is selected from the group consisting of vitamin D3 and vitamin D2. Each possibility represents a separate embodiment of the present invention.

In some embodiments, the non-saturated fatty acid is selected from the group consisting of Omega 3 fatty acids, Omega 6 fatty acids and a combination thereof.

In some embodiments, the growth medium further comprises at least one antimicrobial peptide (AMP) preventing contamination of the cultured cells.

The cell culture comprising a plurality of non-human-animal cell types may be produced by any method as is known in the art. In some embodiments, the cells are bovine cells. After the bovine cells are grown in optimal and efficient conditions, they are used for the production of cultured meat portions as described herein. The cultured meat portion is a combination of cells (two or more types) grown in a co-culture on at least one 3D scaffold. Initially the cells are seeded as single cells or aggregates on the 3D scaffolds, typically in a set ratio between the cell types. The one or more scaffolds and the cells constitute the final food product (e.g. cultured meat portions) as described herein.

The following examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### EXAMPLES

### Example 1

### Production of a Cultured Meat Portion

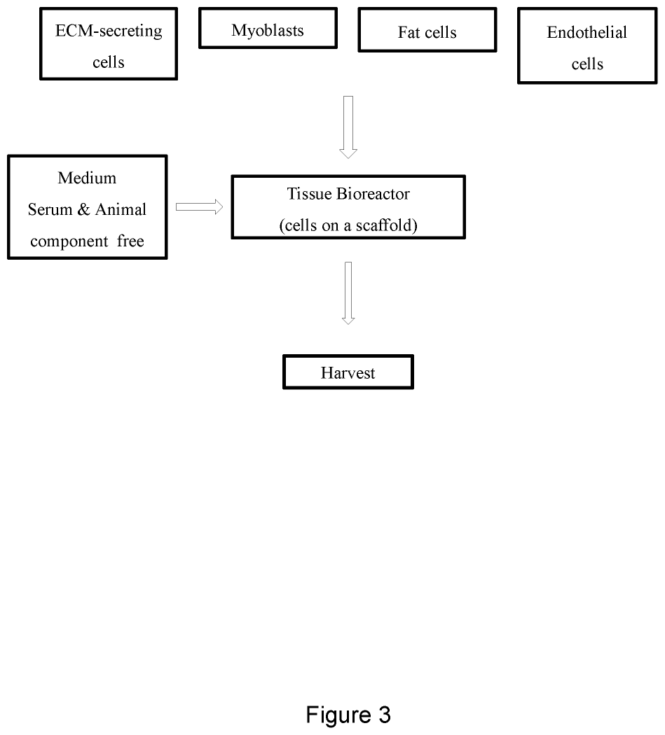

A process flow diagram is depicted in FIG. 3.

Progenitors of bovine-derived ECM-secreting cells, myoblasts, fat cells and endothelial cells that were differentiated from bovine PSC are obtained. The differentiated cells are seeded on a scaffold having a protein content of at least 40% by weight (dry weight) in a cell culture bioreactor according to the present invention and grown in a cultivation system as described herein for 10-14 days. A portion of cultured meat is formed.

The partially differentiated cells are inoculated on the scaffold at a set ratio between the four types of cells and in a certain sequential manner. At the beginning of the production process fresh media (animal component free) is inoculated into the system, and contains specific growth factors and small molecules. Various parameters of the cell culture bioreactor are monitored and adjusted carefully keeping cell viability to optimum level. The temperature (38.6° C.±0.5 ° C.) and pH (6.7-7.2±0.1) of the cell culture bioreactor are maintained. Daily samples of the cell culture bioreactor are collected and analyzed for cell counts (viable and total), medium composition (glucose, ammonia, lactate, osmolality). When Glucose Uptake Rate (GUR) reaches to a maximum level, between 50-500 gr/day, the process ends, and a portion of cultured meat is harvested.

### Example 2

### Cell Growth in Flexible Bag as the Cell Culture Bioreactor of the Invention

2 L sterile bag was designed and produced. The bag was composed of five-layer polyolefin-based TepoFlex®, animal component free film, which provides superior extractables and leachable profiles, water vapor and oxygen barriers, and fluid integrity (produced by Meissner Filtration Products, CA). The bag was sterilized by gamma irradiation (25-40 kGy).

A proof-of-concept experiment was performed in a scale of 70 ml growth media with a single plant-based scaffold having a volume of about 16.5 ml and a single use bag as the cell culture bioreactor.

The 70 ml bag comprising the scaffold was inoculated by 325×10⁶ bovine fibroblasts and myoblasts in a volume of 25 ml growth medium. The head space of the bag was filled with air and the bag was rocked at a speed of 2 cpm, positioned at an angle of 10° at a temperature of 38.5° C. After one hour, additional 45 ml of medium were added. Sample representing the supernatant was taken 2-hour post inoculation for analysis of seeding efficiency. The bag was further incubated statically at a temperature of 38.5° C. and 5% CO₂. Once the measured glucose level was below 4 gr/liter the entire growth medium was refreshed.

Of the 325×10⁶ cells seeded, only 51.3×10⁶ were left in the supernatant after 2 hours. These results indicate that 84% of the cells have been adhered to the scaffold.

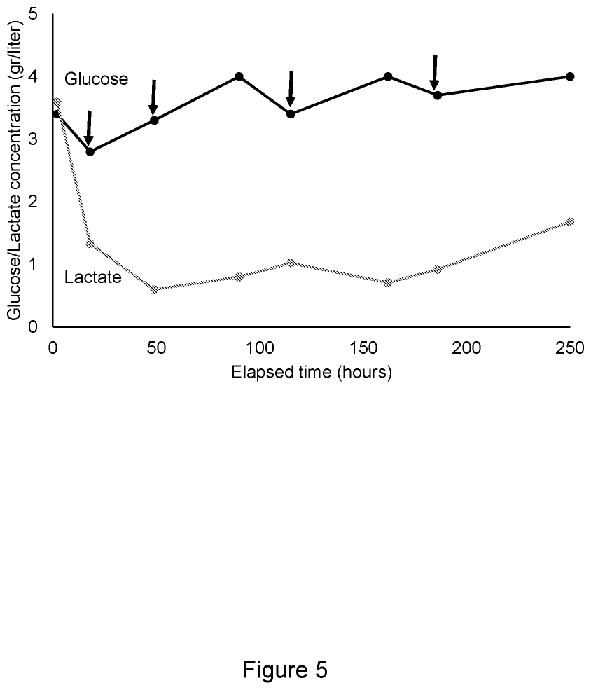

The growth of cells on/in the scaffold with time was followed by analyzing the glucose and lactate levels in the medium. Cell growth is characterized by reduction in the glucose concentration in the medium (indicating glucose uptake by the cells) and by an increase of lactase concentration (indicating metabolic activity of the cells). As is demonstrated in FIG. 5, cells grown on/in the scaffold consumed glucose and produced lactate, indicating that the cells are viable along the first 250 hours examined.

### Example 3

### Growth of Two Cell Types on the Scaffold Within the Cell Culture Bioreactor

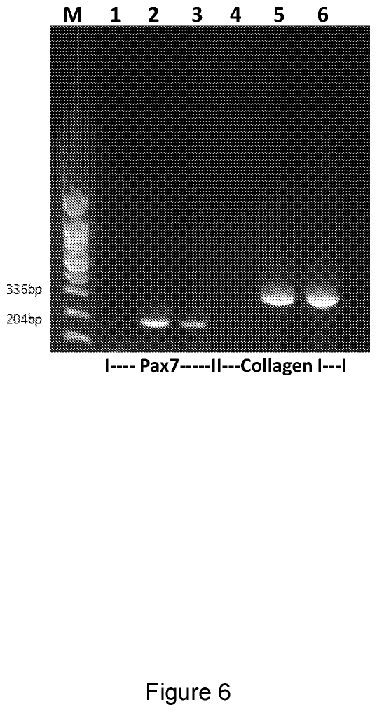

The presence of the fibroblasts and myoblasts seeded on the scaffold at the end of the growth period was examined to ensure that the cultivation system can support growth of more than one cell type. PCR-assistant detection was used. Gene expression of Pax7, a muscle progenitor cells marker, and Collagen type 1, a fibroblasts marker, was tested. Scaffold samples (weight=150 mg) originated from to opposite ends of the scaffold were collected and homogenized and RNA extraction was performed using EZ RNA kit (Biological Industries, Israel). Bare scaffolds with no seeded cells served as negative control. As shown in FIG. 6, Pax7 and Collagen 1 were expressed in both ends of the seeded scaffold. As expected, these markers were not detected in samples obtained from bare scaffold without cells. These data indicate that both the bovine muscle progenitor cells and fibroblasts cells seeded on the scaffold have adhered to and grew concomitantly on the scaffold. Moreover, by showing that the cells are present at opposite ends of the scaffold it is demonstrated that the cells were distributed throughout the entire area of the scaffold.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1-74. (canceled)

75. A cultivation system for producing a cultured food product, comprising:
a. one or more cell culture bioreactors containing therein two or more types of non-human-animal adherent cells seeded on at least one three-dimensional porous edible scaffold; and
b. a delivery system configured to deliver a medium into the one or more cell culture bioreactors in a controlled flow rate, wherein the flow rate is adjusted to nourish the cells seeded on the at least one three-dimensional porous edible scaffold.

76. The system of claim 75, wherein the controlled flow rate is adjusted to at least one of (a) prevent bubble formation in the one or more cell culture bioreactors; and (b) enable circulation of the medium within each of the one or more of cell culture bioreactors in a plug-flow manner.

77. The system of claim 75, wherein the system further comprising at least one of:
a. one or more rockers for radially mixing the medium in the one or more cell culture bioreactors;
b. one or more temperature-control elements for controlling the temperature within the one or more cell culture bioreactors; and
c. one or more sensors for measuring in the medium a parameter selected from the group consisting of liquid level, temperature, pH, dissolved oxygen, concentration of one or more nutrients, concentration of one or more undesired compounds, and any combination thereof.

78. The system of claim 75, wherein the system comprises a plurality of cell culture bioreactors, each of the plurality of cell culture bioreactors separately receiving the medium via the delivery system at a controlled flow rate.

79. The system of claim 75, further comprising:
i. a medium reservoir for supplying a cell growth medium into the bioreactor system;
ii. a treatment vessel configured to: receive the medium; measure in the medium a parameter selected from the group consisting of liquid level, temperature, pH, dissolved oxygen, concentration of one or more nutrients, concentration of one or more undesired compounds, and any combination thereof; and adjust the at least one parameter based on the measurement; and optionally
iii. a dialysis system having a dialyzer and a dialysate, configured to remove undesired compounds from the medium;
wherein the delivery system is further configured to circulate the medium from the one or more cell culture bioreactors into the treatment vessel and/or to circulate the medium from the treatment vessel into the one or more cell culture bioreactors, optionally to circulate the medium from the one or more cell culture bioreactor or the treatment vessel into the dialysis system and subsequently into the treatment vessel.

80. The system of claim 75, wherein the at least one three-dimensional porous edible scaffold comprises a protein content of at least 10% by weight based on the dry weight of the scaffold.

81. The system of claim 75, wherein the two or more types of non-human-animal adherent cells are selected from the group consisting of stromal cells, endothelial cells, fat cells, muscle cells, hepatocytes, cardiomyocytes, renal cells, lymphoid cells, epithelial cells, neural cells, ciliated epithelial cells, gut cells, extracellular matrix (ECM)-secreting cells progenitors thereof, and any combination thereof.

82. The system of claim 75, wherein the cultured food product is cultured meat.

83. The system of claim 75, wherein the cell culture bioreactor is a flexible bag.

84. A cell culture bioreactor for producing a cultured food product, wherein the cell culture bioreactor is in the form of a flexible bag comprising at least one inlet port and at least one outlet port allowing flow of a medium in and out of the bag, wherein the inner face of the flexible bag is of a food-safe material, the bag containing therein at least one three-dimensional porous edible scaffold wherein the scaffold is capable of providing an adherent surface for animal adherent cells.

85. The cell culture bioreactor of claim 84, wherein the at least one three-dimensional porous edible scaffold comprises a protein content of at least 10% by weight based on the dry weight of the scaffold.

86. The cell culture bioreactor of claim 84, wherein the flexible bag is composed of a material selected from the group consisting of a material protecting light-sensitive materials from exposure to light, a material essentially impermeable to water vapors and/or oxygen, and a combination thereof.

87. The cell culture bioreactor of claim 84, wherein the flexible bag is configured for single use to allow sealing of the bag at a time point selected from the group consisting of (a) following insertion of the at least one three-dimensional porous edible scaffold to the cell culture bioreactor and (b) following growth of cells on the at least one three-dimensional porous edible scaffold and production of a cultured food product.

88. The cell culture bioreactor of claim 84, wherein the cultured food product is cultured meat.

89. A packaged food product comprising:
a. a sealed sterile bag having an inner face of a food-safe material; and
b. a cultured meat portion within the bag, substantially filling the entire inner volume of the bag, the cultured meat portion comprising a cellular tissue comprising a plurality of non-human-animal adherent cell types attached to at least one three-dimensional porous edible scaffold.

90. The packaged food of claim 89, wherein the at least one three-dimensional porous edible scaffold comprises a protein content of at least 10% by weight based on the dry weight of the scaffold.

91. The packaged food product of claim 89, wherein the plurality of animal adherent cell types comprises cells selected from stromal cells, endothelial cells, fat cells, muscle cells, hepatocytes, cardiomyocytes, renal cells, lymphoid cells, epithelial cells, neural cells, ciliated epithelial cells, gut cells, extracellular matrix (ECM)-secreting cells progenitors thereof, and any combination thereof.

92. A method for producing a cultured food product on a commercial scale, comprising:
a. seeding two or more types of non-human-animal adherent cells on at least one scaffold disposed within a cell culture bioreactor comprising a cell growth medium, wherein the scaffold is a three-dimensional porous edible scaffold;
b. delivering the cell growth medium into the cell culture bioreactor in a controlled flow rate and adjusting the flow rate to nourish the cells seeded on the at least one three-dimensional porous edible scaffold; and
c. growing the cells until a desired tissue mass is obtained, thereby obtaining a cultured food product.

93. The method of claim 92, further comprising circulating the cell growth medium from the cell culture bioreactor to a treatment vessel and/or dialysis system and subsequently back into the cell culture bioreactor.

94. The method of claim 92, wherein the at least one three-dimensional porous edible scaffold comprises a protein content of at least 10% by weight based on the dry weight of the scaffold.

95. The method of claim 92, further comprising sampling the cell growth medium and measuring the concentration of glucose and/or lactase in the cell growth medium, wherein the cells are grown until glucose uptake rate (GUR) becomes substantially constant.

96. The method of claim 92, wherein the cell culture bioreactor is in the form of a flexible bag for single use, and wherein the method further comprises sealing the bag after the cells reach the desired tissue mass to obtain a packaged food product, the packaged food product comprising the sealed bag and the cultured food product within the bag.

97. A cultured food product comprising a cellular tissue comprising a plurality of non-human-animal adherent cell types attached to at least one three-dimensional porous edible scaffold produced by the method of claim 92.
